(19) United States
(12) Patent Application Publication
(10) Pub. No.: US 2015/0230692 A1
(43) Pub. Date: Aug. 20, 2015

(54) ELECTRONIC ENDOSCOPE

(71) Applicant: OLYMPUS CORPORATION, TOKYO (JP)

(72) Inventors: Eiji MATSUDA, Sagamihara-shi (JP); Keisuke HATANO, Tokyo (JP)

(73) Assignee: OLYMPUS CORPORATION, TOKYO (JP)

(21) Appl. No.: 14/705,187

(22) Filed: May 6, 2015

Related U.S. Application Data
(63) Continuation of application No. PCT/JP2014/058421, filed on Mar. 26, 2014.

(30) Foreign Application Priority Data
Apr. 12, 2013 (JP) 2013-084303

(51) Int. Cl. A61B 1/00; A61B 1/005; A61B 1/07; A61B 1/05; A61B 1/018

(52) U.S. Cl. CPC A61B 1/00114; A61B 1/05; A61B 1/018; A61B 1/00096; A61B 1/07; A61B 1/005

(57) ABSTRACT

An electronic endoscope includes a plurality of small signal cables that extend from an image pickup unit and are inserted through and disposed in an inner space formed in an insertion portion, wherein the plurality of small signal cables are covered by a metal sheath and thereby brought together to form a single signal cable bundle and are arranged as the single signal cable bundle inside an endoscope cable as far as an operation portion, are arranged inside the operation portion in a state in which the metal sheath is stripped off therefrom inside the operation portion, and the plurality of small signal cables protruding from the metal sheath which is stripped off are arranged in a slackened state inside the operation portion.

### CROSS REFERENCE TO RELATED APPLICATION

This application is a continuation application of PCT/JP2014/058421 filed on Mar. 26, 2014 and claims benefit of Japanese Application No. 2013-084303 filed in Japan on Apr. 12, 2013, the entire contents of which are incorporated herein by this reference.

### BACKGROUND OF THE INVENTION

1. Field of the Invention

The present invention relates to an electronic endoscope in which an image pickup apparatus is provided in a distal end portion of an insertion portion.

2. Description of the Related Art

Some conventional electronic endoscopes include an image pickup apparatus that forms an observation image of an object for which light was collected by an objective optical system provided in a distal end portion of an insertion portion to thereby pick up an image of the object.

As disclosed, for example, in Japanese Patent Application Laid-Open Publication No. 2006-55531, in the aforementioned conventional electronic endoscope, a signal cable bundle as an image pickup cable that transmits electrical power and driving signals to the image pickup apparatus is arranged from an insertion portion to an endoscope connector that is connected to an external device such as a video processor through the main body and a composite cable or the like.

A plurality of signal system cables and electrical power system cables provided in the aforementioned conventional electronic endoscope are formed into a single signal cable bundle in which a metal shield layer is provided as a measure for ensuring electromagnetic compatibility (EMC), and the single signal cable bundle is arranged as far as the image pickup apparatus. That is, the plurality of signal system cables and electrical power system cables are bundled together and arranged in the electronic endoscope as a single signal cable bundle that is integrally covered by the metal shield layer.

### SUMMARY OF THE INVENTION

An electronic endoscope according to one aspect of the present invention includes: an insertion portion in which, in order from a distal end thereof, a distal end portion in which an image pickup unit is contained and a metal cylindrical member are connected and made electrically conductive with each other; a plurality of small signal cables that extend from the image pickup unit and are inserted through and disposed in an inner space formed in the insertion portion; an operation portion that is connected to the insertion portion and that includes therein a metal frame that is electrically conductive with the metal cylindrical member; an endoscope cable having a first end portion that is connected to the operation portion; and an endoscope connector that is provided at a second end portion of the endoscope cable and that is to be connected to an external device; wherein the plurality of small signal cables are covered by a metal sheath that is electrically connected to the metal frame and thereby brought together to form a single signal cable bundle and are arranged as the single signal cable bundle inside the endoscope cable as far as the operation portion, the metal sheath is arranged inside the operation portion in a state in which the metal sheath is stripped off inside the operation portion, and the plurality of small signal cables protruding from the metal sheath which is stripped off are arranged in a slackened state inside the operation portion.

According to the present invention that is described above, an electronic endoscope can be provided that ensures electromagnetic compatibility (EMC) and in which a plurality of cables are efficiently arranged so as not to buckle inside a bending portion.

### BRIEF DESCRIPTION OF THE DRAWINGS

FIG. 1 is a perspective view illustrating the configuration of an electronic endoscope according to the present embodiment;

FIG. 2 is a partial cross-sectional view illustrating the configuration of an insertion portion according to the present embodiment;

FIG. 3 is a partial cross-sectional view illustrating the configuration of the insertion portion for mainly describing a treatment instrument channel according to the present embodiment;

FIG. 4 is a partial cross-sectional view illustrating the configuration of a distal end portion for mainly describing illumination means according to the present embodiment;

FIG. 5 is a partial cross-sectional view illustrating the configuration of a proximal end portion of a flexible tube portion according to the present embodiment;

FIG. 6 is a partial cross-sectional view of an operation portion according to the present embodiment;

FIG. 7 is a perspective view for describing a rectangular claw portion that is formed in a pipe sleeve according to the present embodiment;

FIG. 8 is a perspective view for describing a claw portion having an arc shape that is formed in a pipe sleeve according to the present embodiment;

FIG. 9 is a perspective view for describing an approximately hemispherical protruding portion that is formed in an inner circumferential portion of a pipe sleeve according to the present embodiment;

FIG. 10 is a cross-sectional view of the insertion portion along a line X-X in FIG. 2 according to the present embodiment;

FIG. 11 is a cross-sectional view of the insertion portion along a line XI-XI in FIG. 2 according to the present embodiment;

FIG. 12 is a cross-sectional view of the insertion portion along a line XII-XII in FIG. 2 according to the present embodiment;

FIG. 13 is a cross-sectional view of an insertion portion according to another aspect that corresponds to the line X-X in FIG. 2, and which illustrates a first modification of the present embodiment;

FIG. 14 is a cross-sectional view of the insertion portion according to the other aspect that corresponds to the line XI-XI in FIG. 2, and which illustrates the first modification of the present embodiment;

FIG. 15 is a cross-sectional view of the insertion portion according to the other aspect that corresponds to the line XII-XII in FIG. 2, and which illustrates the first modification of the present embodiment;

FIG. 16 is a cross-sectional view of a bending portion according to another aspect, and which illustrates a second modification of the present embodiment;

FIG. 17 is a cross-sectional view of the insertion portion according to the other aspect that corresponds to the line XI-XI in FIG. 2, and which illustrates the second modification of the present embodiment;

FIG. 18 is a cross-sectional view of the insertion portion according to the other aspect that corresponds to the line XII-XII in FIG. 2, and which illustrates the second modification of the present embodiment;

FIG. 19 is a cross-sectional view of an insertion portion according to another aspect that corresponds to the line X-X in FIG. 2, and which illustrates a third modification of the present embodiment;

FIG. 20 is a cross-sectional view of a bending portion according to the other aspect, and which illustrates the third modification of the present embodiment;

FIG. 21 is a perspective view illustrating the configuration of a distal end portion that relates to a first reference example;

FIG. 22 is a front view illustrating the configuration of the distal end portion that relates to the first reference example;

FIG. 23 is a side view illustrating the configuration of the distal end portion that relates to the first reference example;

FIG. 24 is a side view of the distal end portion in a state in which a treatment instrument has been led out from an opening portion that relates to the first reference example;

FIG. 25 is a perspective view illustrating two cylindrical members that are to be fitted together and connected that relates to a second reference example;

FIG. 26 is a cross-sectional view of the two cylindrical members that have been fitted together that relates to the second reference example;

FIG. 27 is a cross-sectional view of the two cylindrical members along a line XXVII-XXVII in FIG. 26, that relates to the second reference example;

FIG. 28 is a perspective view illustrating two cylindrical members having a configuration in which connecting end faces are diagonally cut that relates to the second reference example;

FIG. 29 is a cross-sectional view illustrating two cylindrical members having a configuration in which connecting end faces are diagonally cut that relates to the second reference example;

FIG. 30 is a perspective view illustrating two cylindrical members having a configuration in which connecting end faces are cut in a stepped shape that relates to the second reference example; and

FIG. 31 is a cross-sectional view illustrating two cylindrical members having a configuration in which connecting end faces are cut in a stepped shape that relates to the second reference example.

### DETAILED DESCRIPTION OF THE PREFERRED EMBODIMENT

An embodiment of the present invention is described hereunder with reference to the drawings.

Note that, in the following description, drawings based on the embodiment are schematic ones in which the relationship between the thickness and width of each portion, the thickness ratios between the respective portions and the like are different from those of actual portions, and the drawings may include portions in which the dimensional relationships and ratios are different from those in other drawings.

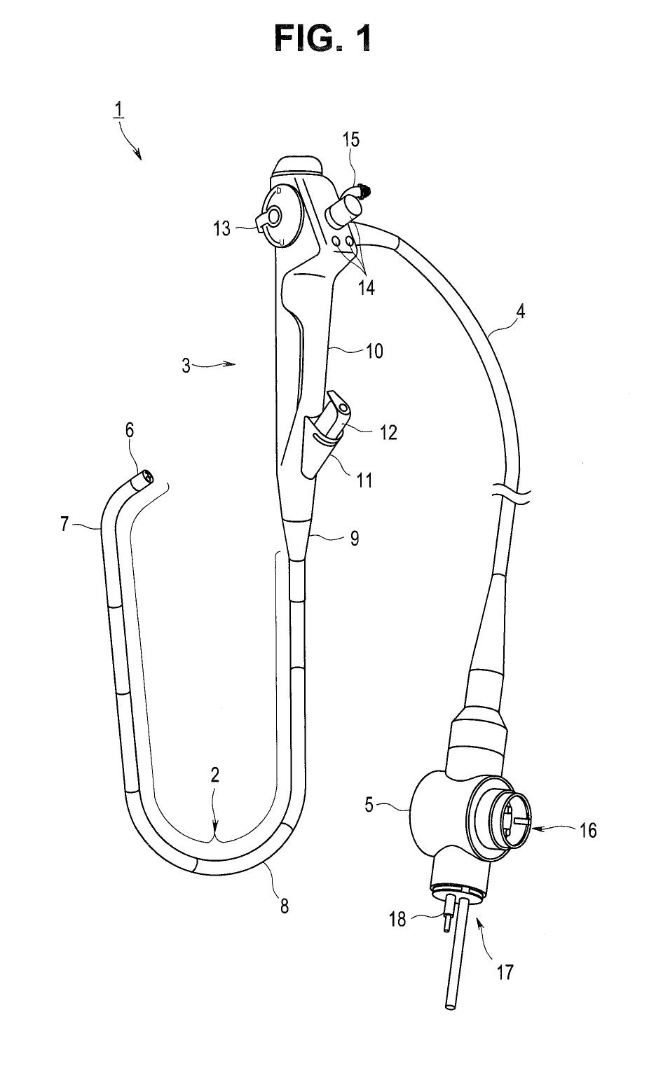

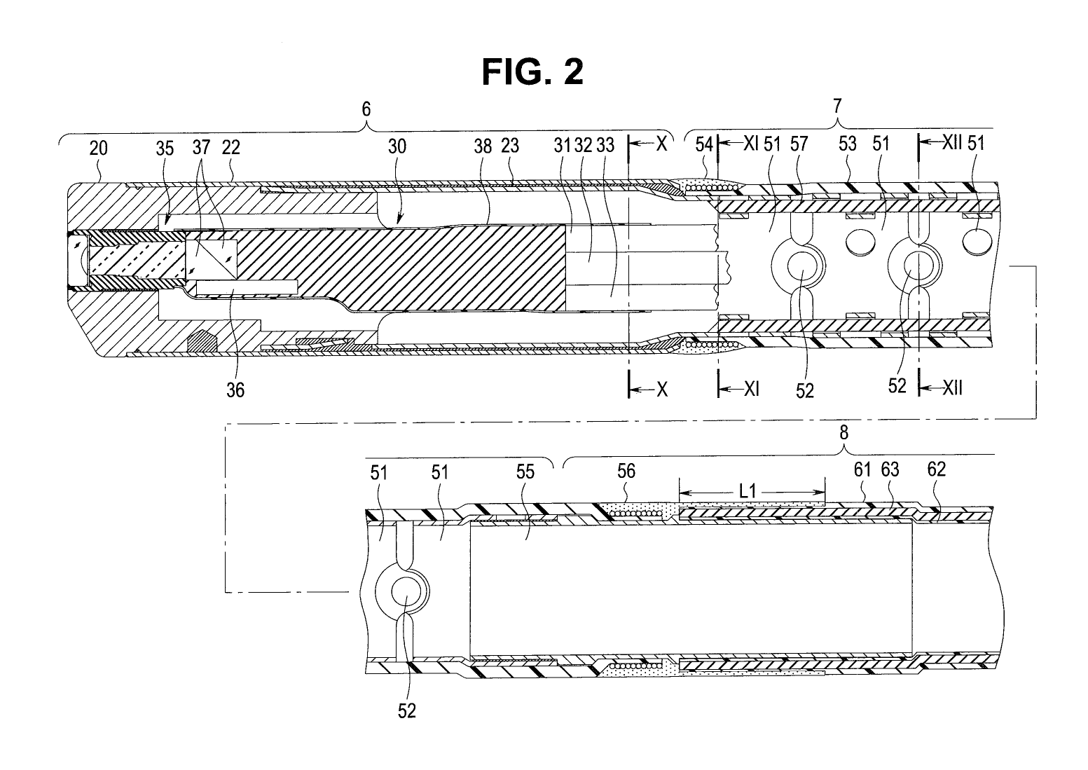

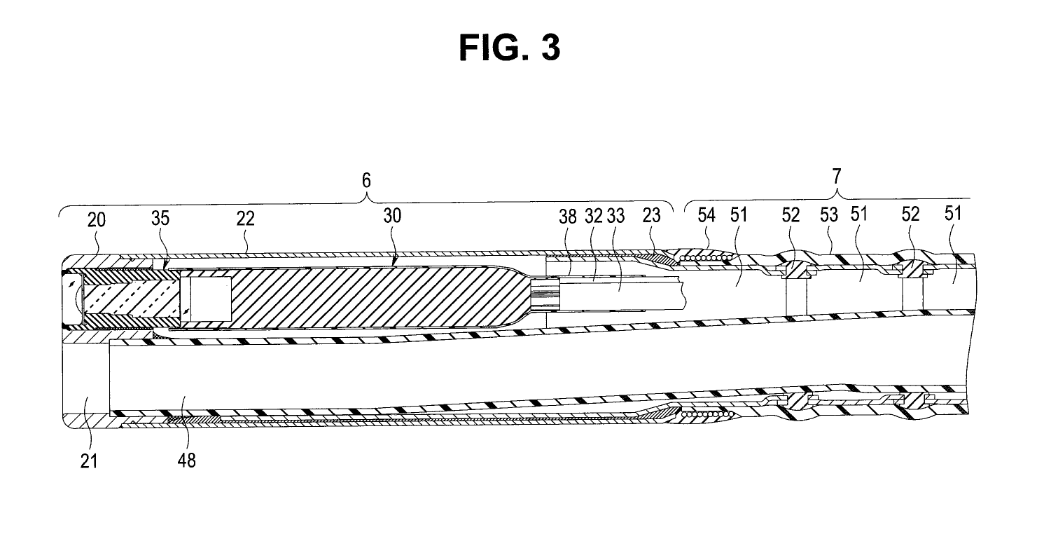

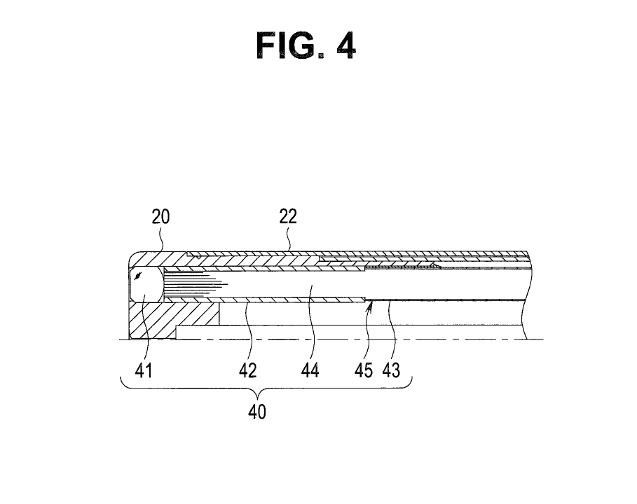

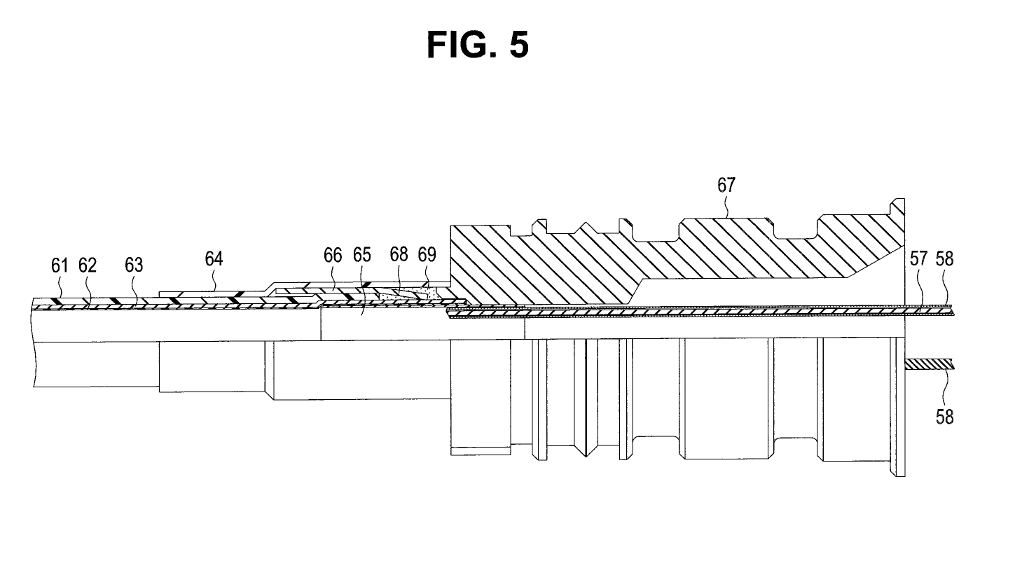

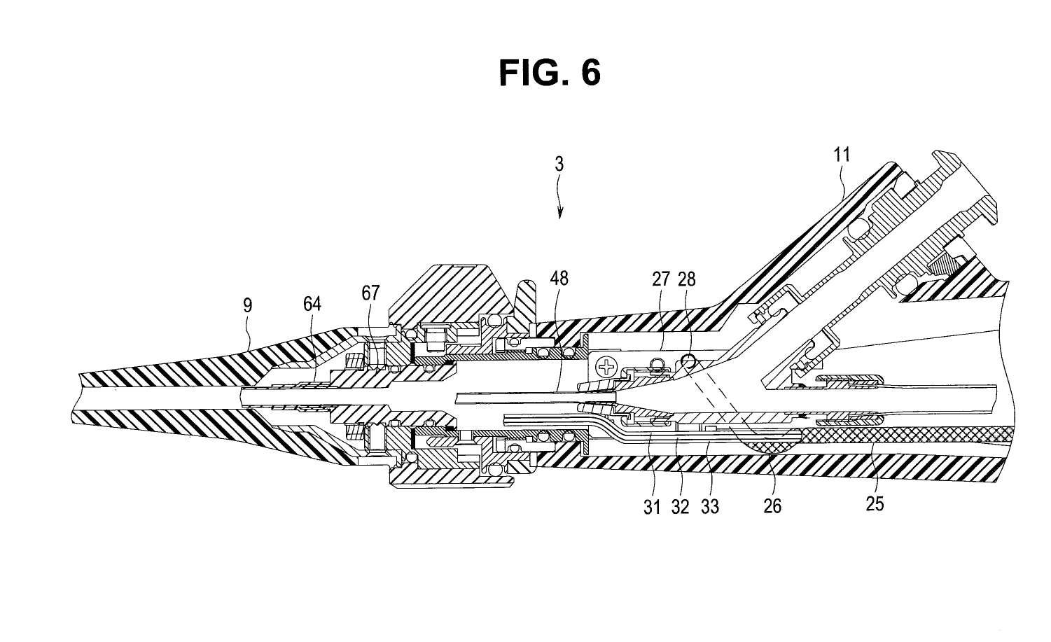

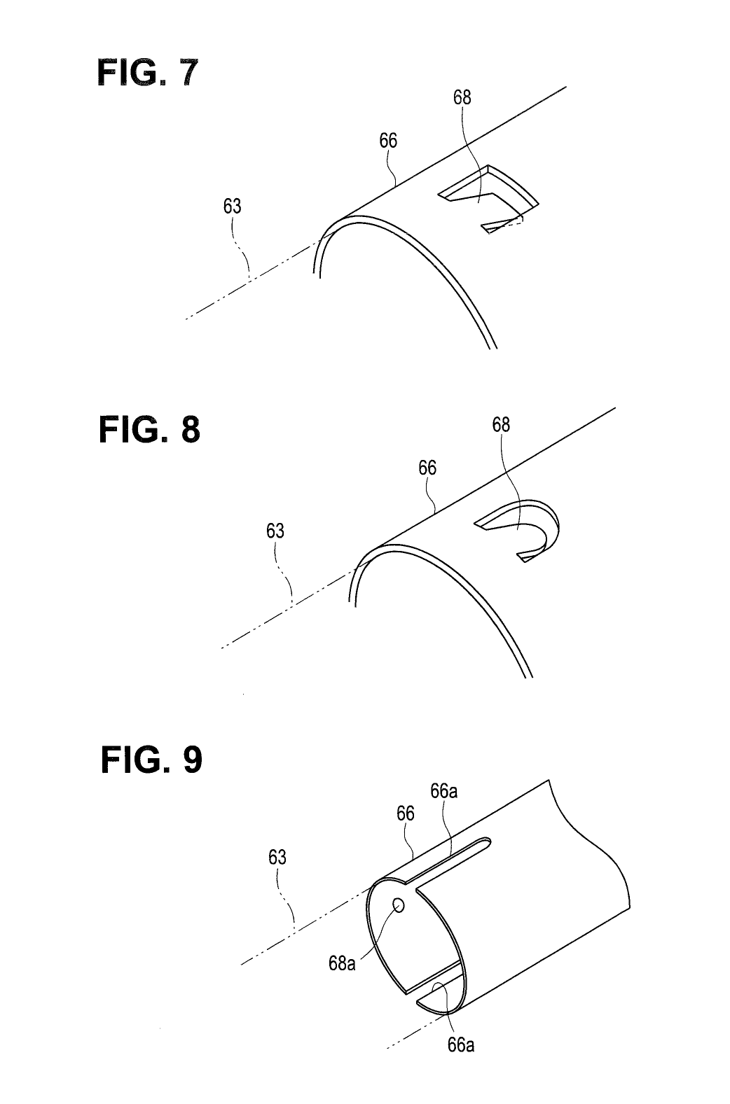

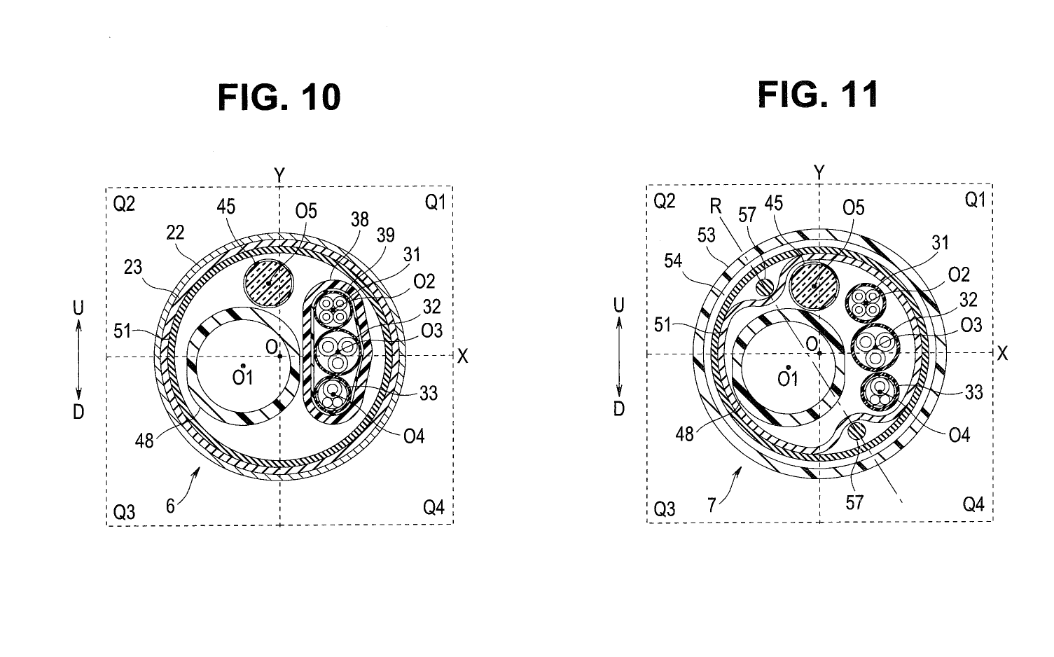

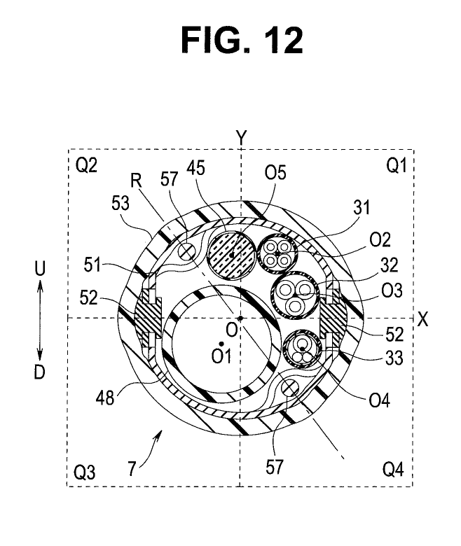

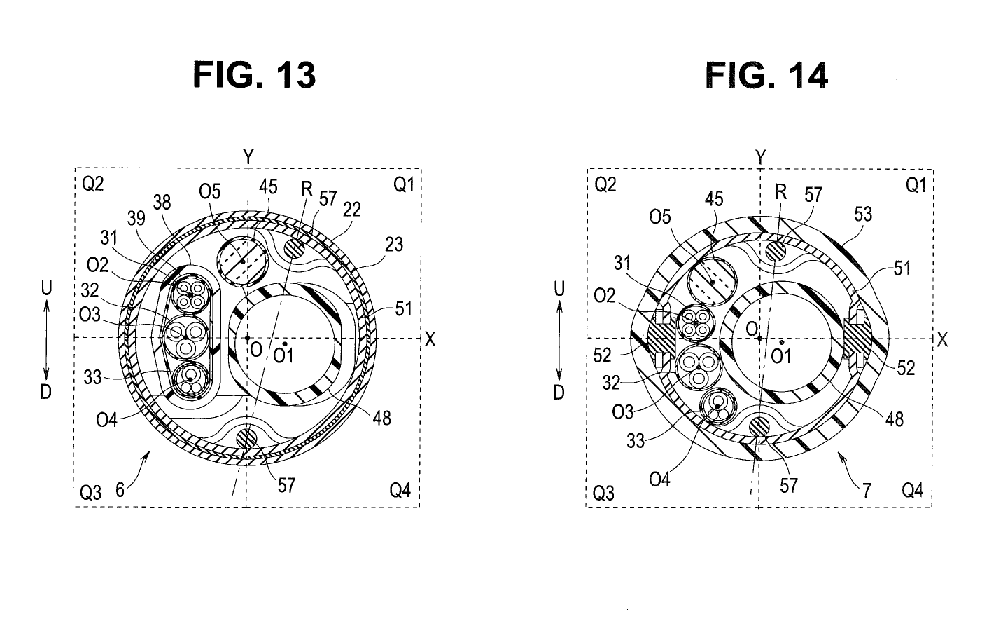

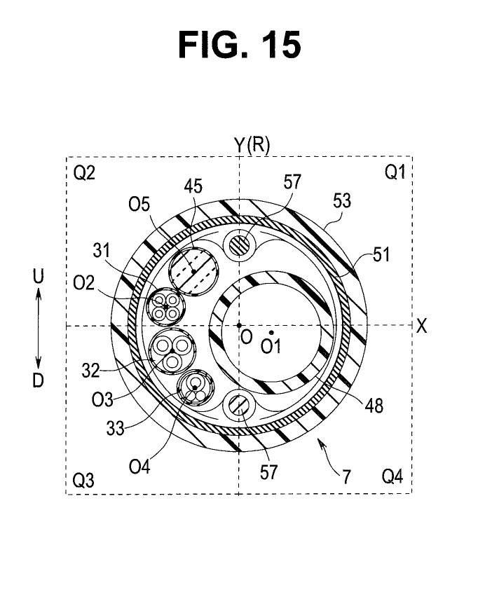

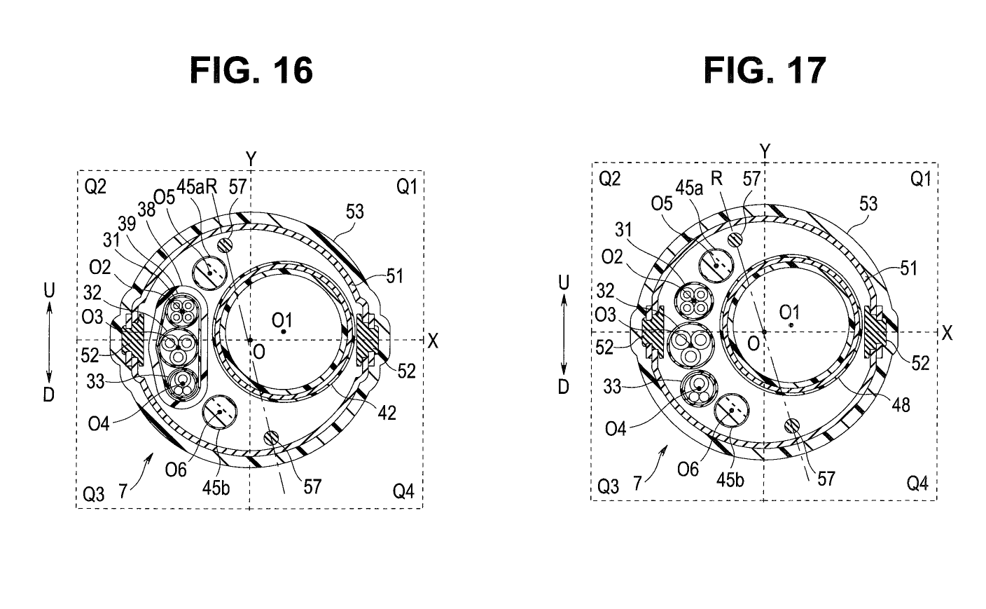

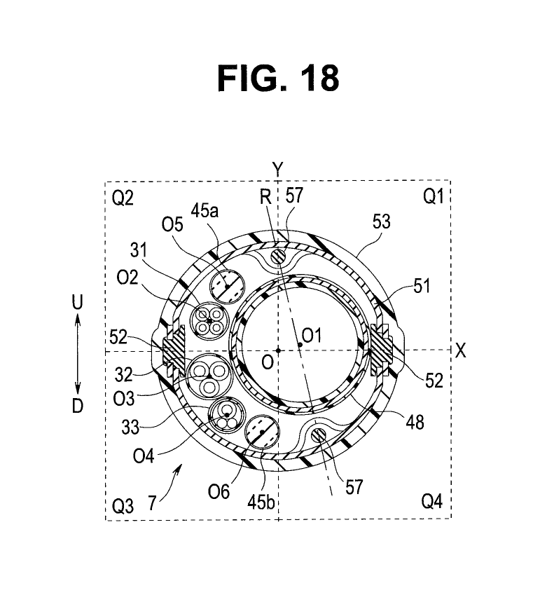

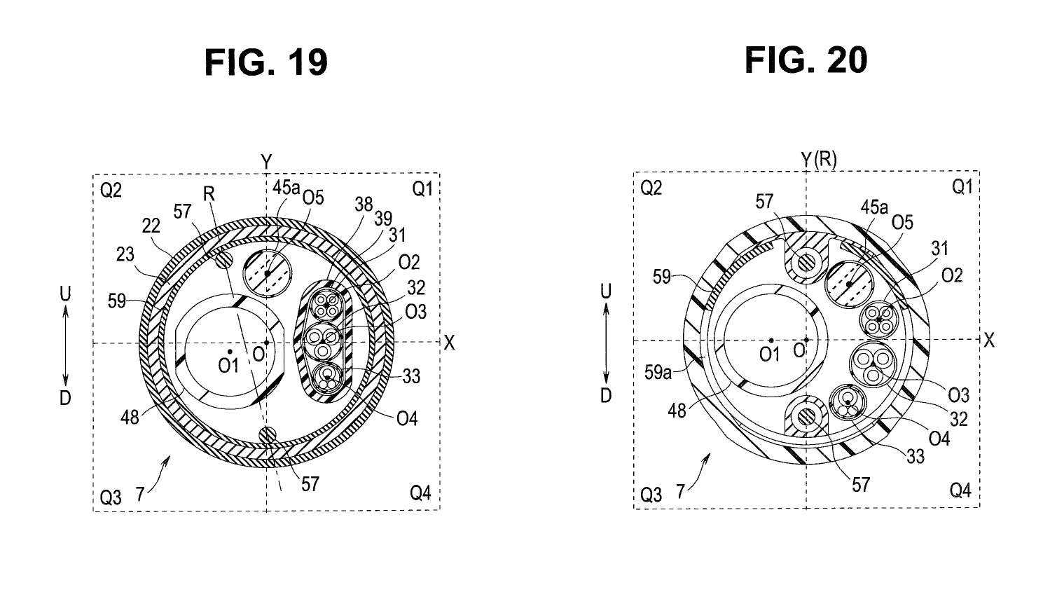

The drawings relate to the present invention. FIG. 1 is a perspective view illustrating the configuration of an electronic endoscope. FIG. 2 is a partial cross-sectional view illustrating the configuration of an insertion portion. FIG. 3 is a partial cross-sectional view illustrating the configuration of the insertion portion for mainly describing a treatment instrument channel. FIG. 4 is a partial cross-sectional view illustrating the configuration of a distal end portion for mainly describing illumination means. FIG. 5 is a partial cross-sectional view illustrating the configuration of a proximal end portion of a flexible tube portion. FIG. 6 is a partial cross-sectional view of an operation portion. FIG. 7 is a perspective view for describing a rectangular claw portion that is formed in a pipe sleeve. FIG. 8 is a perspective view for describing a claw portion having an arc shape that is formed in a pipe sleeve. FIG. 9 is a perspective view for describing an approximately hemispherical protruding portion that is formed in an inner circumferential portion of a pipe sleeve. FIG. 10 is a cross-sectional view of the insertion portion along a line X-X in FIG. 2. FIG. 11 is a cross-sectional view of the insertion portion along a line XI-XI in FIG. 2. FIG. 12 is a cross-sectional view of the insertion portion along a line XII-XII in FIG. 2. FIG. 13 is a cross-sectional view of an insertion portion according to another aspect that corresponds to the line X-X in FIG. 2, and which illustrates a first modification. FIG. 14 is a cross-sectional view of the insertion portion according to the other aspect that corresponds to the line XI-XI in FIG. 2, and which illustrates the first modification. FIG. 15 is a cross-sectional view of the insertion portion according to the other aspect that corresponds to the line XII-XII in FIG. 2, and which illustrates the first modification. FIG. 16 is a cross-sectional view of a bending portion according to another aspect that illustrates a second modification. FIG. 17 is a cross-sectional view of the insertion portion according to the other aspect, and which corresponds to the line XI-XI in FIG. 2, and which illustrates the second modification. FIG. 18 is a cross-sectional view of the insertion portion according to the other aspect that corresponds to the line XII-XII in FIG. 2, and which illustrates the second modification. FIG. 19 is a cross-sectional view of an insertion portion according to another aspect that corresponds to the line X-X in FIG. 2, and which illustrates a third modification. FIG. 20 is a cross-sectional view of a bending portion according to the other aspect, and which illustrates the third modification.

As shown in FIG. 1, an electronic endoscope (hereunder, referred to simply as “endoscope”) 1 of the present embodiment is principally constituted by an insertion portion 2 foamed in a shape of an elongated tube, an operation portion 3 connected to a proximal end of the insertion portion 2, a universal cord 4 that is an endoscope cable that extends from the operation portion 3, and an endoscope connector 5 arranged at a distal end of the universal cord 4.

The insertion portion 2 is a flexible tube-shaped member formed by connecting a distal end portion 6, a bending portion 7 and a flexible tube portion 8 in that order from the distal end side. Of these members, an image pickup unit that is an image pickup apparatus, described later, that contains image pickup means, and illumination means or the like are housed and disposed in the distal end portion 6.

The bending portion 7 is a mechanism region configured to be actively bendable in the two directions of up and down (UP-DOWN) by a rotational operation of a bending lever 13, described later, among operation members of the operation portion 3.

Note that the bending portion 7 is not limited to the aforementioned type, and may be of a type that is bendable in four directions that include the left/right directions in addition to the up/down directions (capable of bending in all circumferential directions, up-down/left-right, around the axis by vertical and horizontal operations).

The flexible tube portion 8 is a tube-shaped member formed with flexibility so as to be passively flexible. In addition to a treatment instrument insertion channel that is described later, various signal wires that are described later that extend from the image pickup apparatus contained in the distal end portion 6 and run from the operation portion 3 to the inside of the universal cord 4, and a light guide that is described later that guides an illuminating light from a light source apparatus and causes the illuminating light to exit from the distal end portion 6 and the like are inserted through the inside of the flexible tube portion 8 (in this case, these members are not illustrated in the drawings).

The operation portion 3 is constituted by: a bend preventing portion 9 that is provided on the distal end side to cover a proximal end of the flexible tube portion 8 and is connected to the flexible tube portion 8; a grasping portion 10 that is connected to the bend preventing portion 9 and which a user manually grasps when using the endoscope 1; operation means for operating various endoscope functions that are provided on an external surface of the grasping portion 10; a treatment instrument insertion portion 11; and a suction valve 15 or the like.

Examples of the operation means provided in the operation portion 3 include the bending lever 13 for performing bending operations of the bending portion 7, and a plurality of operation members 14 for performing air/water feeding operations, suction operations or each operation corresponding to the image pickup means, the illumination means or the like.

The treatment instrument insertion portion 11 includes a treatment instrument insertion port for inserting various kinds of treatment instruments (not shown) and is a component that communicates with a treatment instrument insertion channel through a branching member inside the operation portion 3. A forceps plug 12 that is a cap member for opening/closing the treatment instrument insertion port is arranged in the treatment instrument insertion portion 11. The forceps plug 12 is configured to be detachable (replaceable) with respect to the treatment instrument insertion portion 11.

The universal cord 4 is a composite cable which is inserted through the inside of the insertion portion 2 from the distal end portion 6 of the insertion portion 2 to the operation portion 3, and inside which various signal wires and the like that extend from the operation portion 3 are inserted, and through which are also inserted a light guide of a light source apparatus (not shown) and an air/water feeding tube that extends from an air/water feeding apparatus (not shown).

The endoscope connector 5 includes, on a side face portion, an electrical connector portion 16 to which is connected a signal cable that connects the endoscope 1 with a video processor (not shown) that is an external device, and also includes a light source connector portion 17 to which are connected a light guide bundle, described later, and an electrical cable (not shown) that connect the endoscope 1 with a light source apparatus that is an external device, and an air/water feeding plug 18 that connects the air/water feeding tube (not shown) from the air/water feeding apparatus (not shown) that is an external device.

The configuration of the insertion portion 2 of the endoscope 1 of the present embodiment will now be described based on FIG. 2 to FIG. 5. Note that, in the following description, a description relating to well-known components of the insertion portion 2 is omitted.

As shown in FIG. 2 and FIG. 3, the distal end portion 6 of the insertion portion 2 includes a distal end rigid portion 20 that is a metal block body in which an opening portion 21 is formed in a distal end, and an exterior tube 22 that is fitted and fixed to the exterior of the distal end rigid portion 20.

An image pickup unit 30 that is an image pickup apparatus and illumination means 40 (see FIG. 4) are provided in the distal end rigid portion 20. Further, a treatment instrument channel 48 (see FIG. 3) is fitted and fixed inside the distal end rigid portion 20 so as to communicate with the opening portion 21.

The image pickup unit 30 that is shown in FIG. 2 and FIG. 3 includes a lens unit 35 in which a plurality of objective optical systems are provided, a solid image pickup device 36 as image pickup means, and a prism unit 37 that refracts an observation image of a subject for which light was condensed by the lens unit 35, towards the solid image pickup device.

A plurality of which in this case is three, small signal cables 31, 32 and 33 extend from the proximal end portion of the image pickup unit 30. Note that, since the configuration of the image pickup unit 30 of the present embodiment is similar to the known conventional configuration, a detailed description of the remaining configuration thereof is omitted here.

The three small signal cables 31, 32 and 33 that extend from the image pickup unit 30 are inserted through and disposed inside the insertion portion 2 as described later, and are extended as far as the endoscope connector 5 via the operation portion 3 and the universal cord 4 shown in FIG. 1, and connected to the electrical connector portion 16 of the endoscope connector 5.

The illumination means 40 that is shown in FIG. 4 has a configuration that includes an illumination lens 41 that is fitted and retained in the distal end rigid portion 20, and a plurality of light guides 44 that transmit an illuminating light and that are constituted by bundling a plurality of fibers whose distal end portions are inserted through a tube body 42 that is insertedly fitted in the distal end rigid portion 20 and which are covered by an outer covering 43 at an area on a proximal end side relative to the tube body 42.

Note that the plurality of light guides 44 are inserted through and disposed inside the insertion portion 2 as a light guide bundle 45 in which the light guides 44 are covered by the outer covering 43, and are extended as far as the endoscope connector 5 via the operation portion 3 and the universal cord 4 shown in FIG. 1, and connected to the light source connector portion 17 of the endoscope connector 5.

The treatment instrument channel 48 shown in FIG. 3 is connected so as to communicate with the opening portion 21 of the distal end rigid portion 20, is inserted through and disposed inside the insertion portion 2 and the operation portion 3, and connected to the treatment instrument insertion portion 11 shown in FIG. 1.

In the bending portion 7 of the insertion portion 2, a plurality of bending pieces 51 that are made of metal are rotatably connected by pivoted portions 52 such as rivets, and a bending rubber 53 covers over the outer circumference thereof so as to cover the plurality of bending pieces 51.

The bending piece 51 on the distalmost end side is fitted onto the proximal end portion of the distal end rigid portion 20 of the distal end portion 6 and connected to the distal end rigid portion 20 so as to be electrically conductive therewith.

Note that the aforementioned exterior tube 22 of the distal end portion 6 is arranged so as to cover the bending piece 51 on the distalmost end side, and is fixed by providing an adhesive 23 between the inner circumferential portion of the exterior tube 22 and the outer circumferential portion of the bending piece 51.

A distal end portion of the bending rubber 53 is fixed by means of a bobbin adhesive portion 54 so as to maintain watertightness with an outer circumferential portion of the bending piece 51 on the distalmost end side. A proximal end portion of the bending rubber 53 is fixed by means of a bobbin adhesive portion 56 so as to maintain watertightness with an outer circumferential portion of a connecting tube 55 that is made of metal and that connects the bending piece 51 on the most proximal end side and the flexible tube portion 8.

In this case, the bending portion 7 is configured to be subjected to a bending operation in two directions, namely the upward and downward directions, of an observation image that the image pickup unit 30 picks up, in response to pulling/slackening of two bending operation wires 57 which are connected to the bending piece 51 on the distalmost end side as a result of the plurality of bending pieces 51 in which the two bending operation wires 57 are inserted and held being rotated in a manner that utilizes the pivoted portions 52 as spindles.

The two bending operation wires 57 are inserted through the inside of a coil sheath 58, shown in FIG. 5, in a region extending from the proximal end of the bending portion 7 to the inside of the flexible tube portion 8 and the operation portion 3, and are connected to a sprocket (not shown) that operates in response to operation of the bending lever 13. Note that since a configuration that causes the bending portion 7 to bend is well known, a detailed description of the remaining configuration thereof is omitted here.

As shown in FIG. 2 and FIG. 5, the flexible tube portion 8 of the insertion portion 2 is formed as a flexible tube body having a triple layer structure constituted by an outer-layer resin 61, an inner-layer resin 62 and metal braid 63 of a metal reticular tube that is provided between the outer-layer resin 61 and the inner-layer resin 62. Note that the metal braid may also be formed as a metal helical tube.

The distal end portion of the flexible tube portion 8 is connected to the bending piece 51 on the most proximal end side through the above-described connecting tube 55 that is made of metal. At such time, the metal braid 63 and the connecting tube 55 are connected so as to be electrically conductive with each other.

Note that, because the bending piece 51 on the most proximal end side and the connecting tube 55 are also connected so as to be electrically conductive with each other, the metal braid 63 of the flexible tube portion 8 is electrically conductive with the bending piece 51 on the most proximal end side.

As described above, the distal end portion of the flexible tube portion 8 is fixed to the proximal end portion of the bending rubber 53 by means of a bobbin adhesive portion 56 in a manner such that watertightness is maintained with an outer circumferential portion of the connecting tube 55 that is made of metal and that connects the bending piece 51 on the most proximal end side and the flexible tube portion 8.

In this connection form, the distal end portion of the flexible tube portion 8 is trimmed in the circumferential direction for a predetermined length L1 so that the outer-layer resin 61 has a step (see FIG. 2), and the adhesive of the bobbin adhesive portion 56 is applied onto the portion that was trimmed and fixedly formed thereon.

In a conventional connection form, the outer-layer resin 61 is trimmed and a metal pipe made of stainless steel or the like is covered over the circumference at that position to connect the distal end portion of the flexible tube portion 8 and the proximal end portion of the bending portion 7. However, if the conventional connection form that uses a metal pipe is adopted, in some cases water will leak from a boundary portion between the metal pipe and the outer-layer resin 61. Therefore, to improve on the conventional connection form and ensure that watertightness can be maintained, according to the present embodiment the connection form of the distal end portion of the flexible tube portion 8 and the proximal end portion of the bending portion 7 is a configuration in which the adhesive portion of the bobbin adhesive portion 56 is extended instead of using a metal pipe.

Note that, with this configuration, the wall thickness of the outer-layer resin 61 of the flexible tube portion 8 may be thickened so that adequate watertightness can be maintained even if the outer-layer resin 61 is trimmed in the circumferential direction for the predetermined length L1. Further, because a connecting metal pipe is not used, in addition to thickening the wall of the outer-layer resin 61, it is preferable to increase the hardness of the resin to secure adequate strength.

In addition, although there is a concern that the diameter of the connection portion may be increased as a result of increasing the diameter of the outer-layer resin 61, an increase in the diameter of the connection portion can be prevented by trimming the outer-layer resin 61 and thereafter fixedly forming the adhesive of the bobbin adhesive portion 56 so as to realize the same external diameter as the external diameter of the bending portion 7.

FIG. 5 shows a state in which the proximal end portion of the flexible tube portion 8 is sandwiched between an inner pipe sleeve 65 made of metal that is insertedly provided in the inner-layer resin 62 and a rear pipe sleeve 67 that is made of metal, with the inner pipe sleeve 65 being fitted and fixed inside the rear pipe sleeve 67. Note that a connecting tube portion 66 that is a rear pipe sleeve small-diameter portion formed in an extending manner at the front of the rear pipe sleeve 67 is in a state of being covered by a heat-shrinkable tube 64.

As shown in FIG. 6, the rear pipe sleeve 67 is fixed inside the operation portion 3. Since a configuration for fixing the rear pipe sleeve 67 to the operation portion 3 is similar to a known conventional configuration, a detailed description of the remaining configuration thereof is omitted here.

Note that, as shown in FIG. 5, the proximal end portion of the outer-layer resin 61 of the flexible tube portion 8 is stripped off so that the metal braid 63 is exposed, and the proximal end portion of the metal braid 63 is inserted between the inner pipe sleeve 65 and the rear pipe sleeve 67 so as to contact the inner circumferential portion of the rear pipe sleeve 67.

Further, the proximal end side of the heat-shrinkable tube 64 covers the aforementioned structure so that the connecting tube portion 66 of the rear pipe sleeve 67 that is made of metal is interposed therebetween. A rectangular claw portion 68 (see FIG. 5 and FIG. 7) formed in the connecting tube portion 66 bends in the inner diameter direction so as to contact the metal braid 63 of the flexible tube portion 8.

That is, even if a large clearance arises between the inner diameter of the connecting tube portion 66 of the rear pipe sleeve 67 and the external diameter of the metal braid 63, the claw portion 68 definitely comes in contact with the outer circumferential portion of the metal braid 63, and thus electrical conductivity can be ensured between the metal braid 63 and the rear pipe sleeve 67 via the connecting tube portion 66.

Thus, since the metal braid 63 of the flexible tube portion 8 contacts the inner circumferential portion of the rear pipe sleeve 67, and the claw portion 68 of the connecting tube portion 66 contacts the outer circumferential portion thereof, the configuration is one in which electrical conductivity is reliably achieved between the metal braid 63 and the rear pipe sleeve 67 through the connecting tube portion 66.

Thus, the proximal end portion of the flexible tube portion 8 is fixed in a state in which electrical conductivity is achieved between the metal braid 63 and the rear pipe sleeve 67.

Note that the claw portion 68 of the connecting tube portion 66 of the rear pipe sleeve 67 is not limited to a rectangular shape, and a configuration may also be adopted in which one part of the connecting tube portion 66 is notched in a U-shape to form the claw portion 68 in an arc shape as shown in FIG. 8. Since this claw portion 68 does not have corner parts (edges) in comparison to the rectangular shape, the metal braid 63 that is a metal reticular tube can be prevented from unravelling.

Further, as shown in FIG. 9, an approximately hemispherical protruding portion 68a may be provided in the inner circumferential portion of the connecting tube portion 66 of the rear pipe sleeve 67 instead of the claw portion 68. Note that, in the connecting tube portion 66 of the rear pipe sleeve 67 in this case, two slits 66a are formed for causing the connecting tube portion 66 to change shape so as to expand in the external diameter direction so that the flexible tube portion 8 is not crushed in the inner diameter direction when the connecting tube portion 66 is being fitted onto the metal braid 63. Even when this configuration for contacting the metal braid 63 by means of the approximately hemispherical protruding portion 68a is adopted, since there are few corner parts (edges), it is difficult for unravelling of the metal braid 63 to occur.

As described above, in the insertion portion 2, the distal end rigid portion 20 of the distal end portion 6, the plurality of bending pieces 51 inside the bending portion 7, and the metal braid 63 of the flexible tube portion 8 are electrically conductive with each other, and the metal braid 63 is electrically conductive with the rear pipe sleeve 67 that is provided in the operation portion 3.

Further, similarly to the known configuration, the rear pipe sleeve 67 is made electrically conductive with a metal frame portion 27 (see FIG. 6) provided in the operation portion 3, and the metal frame portion 27 is made electrically conductive with a metal shield (not shown) of the universal cord 4 that is connected to the operation portion 3.

Note that the metal shield of the universal cord 4 is electrically connected to a ground of an external device through the endoscope connector 5.

Accordingly, the endoscope 1 of the present embodiment is configured so that the insertion portion 2 is in a state in which an inner space thereof is electromagnetically shielded by the plurality of cylindrical bending pieces 51 and the cylindrical metal braid 63 from the distal end portion 6 that is a metal block body, and is connected to a ground that is an external device through the operation portion 3 and the universal cord 4.

That is, the endoscope 1 has a configuration in which an inner space is electromagnetically shielded by a plurality of metal cylindrical members from the insertion portion 2 on the distal end side to the endoscope connector 5 that is provided at the proximal end of the universal cord 4.

Next, the configuration of the three small signal cables 31, 32 and 33 that extend from the image pickup unit 30 in the endoscope 1 of the present embodiment as well as the respective arrangement relations between the three small signal cables 31, 32 and 33 and the treatment instrument channel 48 and the light guide bundle 45 will be described hereunder.

First, as shown in FIG. 6, the three small signal cables 31, 32 and 33 that extend from the image pickup unit 30 are covered by a metal sheath 26 and thereby brought together as an electric cable bundle 25 that is inserted through and disposed inside the area from the universal cord 4 to the operation portion 3.

The electric cable bundle 25 is in a state in which a metal shield that bundles together and covers the three small signal cables 31, 32 and 33 is covered by an outer covering sheath (not shown) that is provided on an inner face side, and is stripped off inside the operation portion 3 or the universal cord 4. Further, the small signal cables 31, 32 and 33 including the outer covering sheath (not shown) are covered by the metal sheath 26 that is an ultrathin tube made of metal from the endoscope connector 5, and the end portion of the metal sheath 26 is connected to the metal frame 27 inside the operation portion 3 by a screw 28 so as to be electrically conductive therewith. That is, the electric cable bundle 25 in which the small signal cables 31, 32 and 33 including the outer covering sheath (not shown) are brought together as a single bundle is formed as a result of covering the small signal cables 31, 32 and 33 including the outer covering sheath with the metal sheath 26 from the endoscope connector 5 to the operation portion 3.

The small signal cables 31, 32 and 33 that protrude from the metal sheath 26 are disposed in a slackened state inside the operation portion 3. That is, by slackening the three small signal cables 31, 32 and 33 inside the operation portion 3, the insertion portion 2 is flexible and advancing and retracting movement amounts that arise as the result of bending operations are absorbed and an excessive load does not arise and thus breakage of the three small signal cables 31, 32 and 33 or the like can be prevented.

Note that among the three small signal cables 31, 32 and 33 of the present embodiment, the first small signal cable 31 is a cable for sending and receiving vertical driving signals to the solid image pickup device 36 provided in the image pickup unit 30, the second small signal cable 32 is a cable for sending and receiving horizontal driving signals to the solid image pickup device 36, and the third small signal cable 31 is an electrical power system cable for driving the solid image pickup device 36.

Further, as shown in FIG. 10 to FIG. 12, because the frequency of the signals is high, a thicker signal wire is used for the second small signal cable 32 in comparison to the first small signal cable 31 and the third small signal cable 33, and the external diameter of the second small signal cable 32 is the largest diameter among the three small signal cables 31, 32 and 33.

FIG. 10 shows a state in which, at the proximal end portion of the distal end portion 6, the distal end portion sides of the three small signal cables 31, 32 and 33 that protrude from the image pickup unit 30 are bundled together using a PE tape 39, and are covered by the heat-shrinkable tube 38 provided in the image pickup unit 30.

At such time, the three small signal cables 31, 32 and 33 are arranged in parallel along the upward-downward directions that are the U-D (up-down) directions in which the bending portion 7 bends as indicated by the vertical direction in the drawing, and are disposed in the order of, from the upper side, the first small signal cable 31, the second small signal cable 32 and the third small signal cable 33. That is, among the three small signal cables 31, 32 and 33, the second small signal cable 32 that has the largest diameter is positioned at the center in the vertical direction.

Further, when it is assumed that an orthogonal coordinate system is divided in the vertical and horizontal directions into four quadrants Q1, Q2, Q3 and Q4 by the X-axis and Y-axis in a manner that takes the center of distal end portion 6 that is a center O of the insertion portion 2 as an origin, within the distal end portion 6, a center O1 of the treatment instrument channel 48 having the largest diameter is positioned inside the quadrant Q3 that is a third quadrant in terms of the orthogonal coordinate system, a center O2 of the first small signal cable 31 and a center O3 of the second small signal cable 32 are positioned inside the quadrant Q1 that is a first quadrant in terms of the orthogonal coordinate system, a center O4 of the third signal cable 33 is positioned inside the quadrant Q4 that is a fourth quadrant in terms of the orthogonal coordinate system, and a center O5 of the light guide bundle 45 is positioned inside the quadrant Q2 that is a second quadrant in terms of the orthogonal coordinate system, respectively.

Furthermore, as shown in FIG. 11 and FIG. 12, when it is assumed that an orthogonal coordinate system is divided in the vertical and horizontal directions into four quadrants Q1, Q2, Q3 and Q4 by the X-axis and Y-axis in a manner that takes the center of the bending portion 7 that is the center O of the insertion portion 2 as an origin, within the bending portion 7 also, the center O1 of the treatment instrument channel 48 is positioned inside the quadrant Q3, the center O2 of the first small signal cable 31 and the center O3 of the second small signal cable 32 are positioned the quadrant Q1, the center O4 of the third signal cable 33 is positioned inside the quadrant Q4, and the center O5 of the light guide bundle 45 is positioned inside the quadrant Q2.

In addition, within the bending portion 7 also, the three small signal cables 31, 32 and 33 are, as described above, arranged in parallel along the U-D directions in which the bending portion 7 bends, and are arranged in the order of the first small signal cable 31, the second small signal cable 32 and the third small signal cable 33 from the “up” side. That is, inside the bending portion 7 also, among the three small signal cables 31, 32 and 33, the second small signal cable 32 that has the largest diameter is arranged so as to be at the center in the vertical direction.

Further, inside the bending portion 7, among two regions that are divided by a line R that links the two bending operation wires 57 for performing operations to bend the bending portion 7, the center O1 of the treatment instrument channel 48 is disposed in one of the regions, and the center O2 of the first small signal cable 31, the center O3 of the second small signal cable 32, the center O4 of the third signal cable 33 and the center O5 of the light guide bundle 45 are disposed in the other region.

That is, the endoscope 1 of the present embodiment is configured so that, inside the bending portion 7 of the insertion portion 2, the treatment instrument channel 48 or the light guide bundle 45 that are other built-in elements are not arranged in the upward-downward bending direction of the bending portion 7 with respect to the first small signal cable 31, the second small signal cable 32 and the third signal cable 33.

Thus, in the endoscope 1, in particular, in an orthogonal coordinate system that takes as an origin the center of the bending portion 7 (center O of the insertion portion 2) that bends upward or downward (in the U-D directions) in response to operations that pull or slacken the two bending operation wires 57, the center O2 of the first small signal cable 31, the center O3 of the second small signal cable 32, the center O4 of the third signal cable, or the center O5 of the light guide bundle 45 is disposed in a quadrant (here, the first quadrant Q1 or the fourth quadrant Q4 in terms of the orthogonal coordinate system) obtained by dividing the orthogonal coordinate system along the Y-axis that joins the upward and downward directions that are different quadrants to a quadrant in which the center O1 of the treatment instrument channel 48 is disposed (third quadrant Q3 in terms of the orthogonal coordinate system).

By this means, in the endoscope 1 of the present embodiment, when the bending portion 7 is subjected to a bending operation in the upward or downward direction, in particular, since the first small signal cable 31, the second small signal cable 32 and the third signal cable 33 do not buckle due to being squashed by the treatment instrument channel 48 or the light guide bundle 45, breakage of the three small signal cables 31, 32 and 33 is prevented.

Note that the light guide bundle 45 in which a plurality of fibers are bundled together is disposed above the treatment instrument channel 48 inside the bending portion 7. Consequently, when an operation is performed to bend the bending portion 7 in the upward or downward direction, even if the treatment instrument channel 48 contacts and crushes several fibers among the plurality of fibers and the several fibers break, a problem does not arise since the required illuminating light is obtained.

In addition, with respect to the endoscope 1, if a configuration is adopted in which the plurality of, in this case, three, small signal cables 31, 32 and 33 are separated and not bundled together to form a single cable and are inserted through and disposed inside the insertion portion 2, the diameter of the insertion portion 2 can be reduced since the small signal cables 31, 32 and 33 can be effectively disposed in empty space inside the insertion portion 2.

Furthermore, since the inner space of the insertion portion 2 is approximately circular, reducing the diameter of the insertion portion 2 is facilitated by efficiently disposing the three small signal cables 31, 32 and 33 in the empty space so that the second small signal cable 32 that has the largest diameter is in the center.

Note that, as described above, in the endoscope 1, since the three small signal cables 31, 32 and 33 from which the metal sheath 26 was stripped off from the single electric cable bundle 25 within the operation portion 3 are inserted through and disposed in the insertion portion 2 whose inner space is electromagnetically shielded by the plurality of ring-shaped bending pieces 51 and the cylindrical-shaped metal braid 63 from the distal end portion 6 that is a metal block body, the endoscope 1 has a configuration in which electromagnetic compatibility (EMC) is ensured.

(First Modification)

Next, as a first modification, a configuration of the endoscope 1 is described hereunder in which the three small signal cables 31, 32 and 33, the treatment instrument channel 48 and the light guide bundle 45 are disposed inside the insertion portion 2 in a different manner to the above described configuration.

In this case also, as shown in FIG. 13 to FIG. 15, the three small signal cables 31, 32 and 33 are arranged in parallel along the U-D (up-down) directions in which the bending portion 7 bends as indicated by indicating the vertical direction in the drawing, and similarly to the above described configuration, are disposed in the order of, from the “up” side, the first small signal cable 31, the second small signal cable 32 and the third small signal cable 33. That is, the second small signal cable 32 that has the largest diameter is positioned in the center.

As shown in FIG. 13, the three small signal cables 31, 32 and 33 are disposed inside the distal end portion 6 so that the center O1 of the treatment instrument channel 48 that has the largest diameter is positioned inside a quadrant Q4 that is a fourth quadrant in terms of the orthogonal coordinate system, the center O2 of the first small signal cable 31, the center O3 of the second small signal cable 32 and the center O5 of the light guide bundle 45 are positioned inside a quadrant Q2 that is a second quadrant in terms of the orthogonal coordinate system, and the center O4 of the third signal cable 33 is positioned inside a quadrant Q3 that is a third quadrant in terms of the orthogonal coordinate system, respectively.

Further, as shown in FIG. 14 and FIG. 15, inside the bending portion 7, the center O1 of the treatment instrument channel 48 that has the largest diameter is positioned inside the quadrant Q4, the center 02 of the first small signal cable 31 and the center O5 of the light guide bundle 45 are positioned inside the quadrant Q2, and the center O3 of the second small signal cable 32 and the center O4 of the third signal cable 33 are positioned inside the quadrant Q3.

In addition, in this case as well, within the bending portion 7 also, the three small signal cables 31, 32 and 33 are arranged in parallel along the up-down (U-D) directions in which the bending portion 7 bends, and are arranged in the order, from the upper side, of the first small signal cable 31, the second small signal cable 32 and the third small signal cable 33. Note that, in this case also, among the three small signal cables 31, 32 and 33, the second small signal cable 32 that has the largest diameter is arranged so as to be at the center in the vertical direction.

Further, inside the bending portion 7, among two regions that are divided by a line R that links the two bending operation wires 57 for performing operations to bend the bending portion 7, the center O1 of the treatment instrument channel 48 is disposed in one of the regions, and the center O2 of the first small signal cable 31, the center O3 of the second small signal cable 32, the center 04 of the third signal cable 33 and the center O5 of the light guide bundle 45 are disposed in the other region.

That is, the endoscope 1 of the present modification is configured so that, inside the bending portion 7 of the insertion portion 2, the treatment instrument channel 48 that is another built-in element is not arranged in the upward-downward bending directions of the bending portion 7 with respect to the first small signal cable 31, the second small signal cable 32 and the third signal cable 33.

Thus, in the endoscope 1, in particular, in an orthogonal coordinate system that takes the center of the bending portion 7 of the insertion portion 2 as an origin, the center O2 of the first small signal cable 31, the center O3 of the second small signal cable 32, the center O4 of the third signal cable 33 or the center O5 of the light guide bundle 45 is disposed in a quadrant (here, the third quadrant Q3 or the second quadrant Q2 in terms of the orthogonal coordinate system) obtained by dividing the orthogonal coordinate system along the Y-axis that joins the upward and downward directions that are different quadrants to a quadrant in which the center O1 of the treatment instrument channel 48 is disposed (fourth quadrant Q4 in terms of the orthogonal coordinate system).

Thus, the present modification has a configuration in which the same operations and/or effects as described above are obtained, and in which the three small signal cables 31, 32 and 33 are disposed in a state in which electromagnetic compatibility (EMC) is ensured and breakage of the three small signal cables 31, 32 and 33 when operations are performed to bend the bending portion 7 in the upward and downward directions is prevented, and in which the diameter of the insertion portion 2 can be reduced.

(Second Modification)

Next, as a second modification, a configuration of the endoscope 1 is described hereunder in which two light guide bundles are provided that is different from the above described configuration, that is a configuration in which the three small signal cables 31, 32 and 33, the treatment instrument channel 48 and two light guide bundles 45a and 45b are disposed inside the insertion portion 2.

FIG. 16 shows a state in which, at the distal end portion of the bending portion 7, the distal end portion sides of the three small signal cables 31, 32 and 33 that protrude from the image pickup unit 30 are bundled together using the PE tape 39 and are covered by the heat-shrinkable tube 38 that is provided in the image pickup unit 30.

As shown in FIG. 16 to FIG. 18, the three small signal cables 31, 32 and 33 are arranged in parallel along the U-D (up-down) directions in which the bending portion 7 bends as indicated by the vertical direction in the drawing, and similarly to the above described configuration, are disposed in the order of, from the “up” side, the first small signal cable 31, the second small signal cable 32 and the third small signal cable 33. That is, the second small signal cable 32 that has the largest diameter is positioned in the center.

The three small signal cables 31, 32 and 33 are disposed inside the distal end portion 6 so that the center O1 of the treatment instrument channel 48 that has the largest diameter is positioned inside a quadrant Q1 that is a first quadrant in terms of the orthogonal coordinate system, the center O2 of the first small signal cable 31 and a center O5 of the first light guide bundle 45a are positioned inside a quadrant Q2 that is a second quadrant in terms of the orthogonal coordinate system, and the center O3 of the second small signal cable 32, the center O4 of the third signal cable 33 and a center O6 of the second light guide bundle 45b are positioned inside a quadrant Q3 that is a third quadrant in terms of the orthogonal coordinate system, respectively.

In addition, in this case as well, within the bending portion 7 also, the three small signal cables 31, 32 and 33 are arranged in parallel along the U-D directions in which the bending portion 7 bends, and are arranged in the order of, from the “up” side, the first small signal cable 31, the second small signal cable 32 and the third small signal cable 33. Note that, in this case also, the second small signal cable 32 that has the largest diameter is arranged so as to be at the center.

Further, in this configuration also, inside the bending portion 7, among two regions that are divided by a line R that links the two bending operation wires 57 for performing operations to bend the bending portion 7, the center O1 of the treatment instrument channel 48 is disposed in one of the regions, and the center O2 of the first small signal cable 31, the center O3 of the second small signal cable 32, the center O4 of the third signal cable 33, the center O5 of the first light guide bundle 45a and the center O6 of the second light guide bundle 45b are disposed in the other region.

That is, the endoscope 1 of the present modification is configured so that, inside the bending portion 7 of the insertion portion 2, the treatment instrument channel 48 that is another built-in element is not arranged in the upward-downward bending directions of the bending portion 7 with respect to the first small signal cable 31, the second small signal cable 32 and the third small signal cable 33.

Thus, in the endoscope 1 of the present modification, in particular, inside the bending portion 7 of the insertion portion 2 that bends upward or downward in response to an operation that pulls or slackens the two bending operation wires 57, with respect to an orthogonal coordinate system that takes the center of the bending portion 7 (center O of the insertion portion 2) as an origin, the center O2 of the first small signal cable 31, the center O3 of the second small signal cable 32, the center O4 of the third signal cable 33, the center 05 of the first light guide bundle 45a or the center O6 of the second light guide bundle 45b is disposed in a quadrant (here, the third quadrant Q3 or the second quadrant Q2 in terms of the orthogonal coordinate system) obtained by dividing the orthogonal coordinate system along the Y-axis that joins the upward and downward directions and that are different quadrants to a quadrant in which the center O1 of the treatment instrument channel 48 is disposed (first quadrant Q1 in terms of the orthogonal coordinate system).

Thus, the endoscope 1 of the present modification also has a configuration in which the same operations and/or effects as described above are obtained, and in which the three small signal cables 31, 32 and 33 are disposed in a state in which electromagnetic compatibility (EMC) is ensured and breakage of the three small signal cables 31, 32 and 33 when operations are performed to bend the bending portion 7 in the upward and downward directions is prevented, and in which the diameter of the insertion portion 2 can be reduced.

As described in the foregoing, in the endoscope 1 of the present embodiment, in particular, inside the bending portion 7 of the insertion portion 2, with respect to the quadrants of the orthogonal coordinate system that takes the center of the bending portion 7 (center O of the insertion portion 2) as an origin, by arranging at least the three small signal cables 31, 32 and 33 in a separated manner in quadrants obtained by dividing the orthogonal coordinate system along the Y-axis that joins the upward and downward directions and that are different quadrants to a quadrant in which the center O1 of the treatment instrument channel 48 that has the largest diameter among the built-in elements in the upward-downward bending directions of the bending portion 7 is disposed, breakage of the three small signal cables 31, 32 and 33 can be prevented and the diameter of the insertion portion 2 can be reduced.

Furthermore, with respect to the quadrants of the orthogonal coordinate system that takes the center of the bending portion 7 (center O of the insertion portion 2) as an origin, besides the treatment instrument channel 48, it is preferable that the three small signal cables 31, 32 and 33 are disposed in quadrants obtained by dividing the orthogonal coordinate system along the Y-axis that joins the upward and downward directions and that are different quadrants to a quadrant in which the center O5 of the light guide bundle 45a and the center O6 of the light guide bundle 45b that are other built-in elements are disposed.

(Third Modification)

Note that, as shown in FIG. 19 and FIG. 20, the bending portion 7 provided in the insertion portion 2 of the endoscope 1 may have a configuration including a bending tube 59 in which a plurality of slits 59a (see FIG. 20) are formed in the longitudinal direction in a super-elastic alloy pipe is provided instead of the plurality of bending pieces 51 that are rotatably connected by pivoted portions 52 such as rivets that are provided inside the bending portion 7.

That is, unlike the respective configurations described above, the bending portion 7 in this case has a configuration in which the pivoted portions 52 are not provided, and the bending tube 59 that is formed so that a plurality of metal cylindrical members are connected in the longitudinal axis direction by the plurality of slits 59a is contained therein.

According to the endoscope 1 of the present modification, since the arrangement of the three small signal cables 31, 32 and 33, the treatment instrument channel 48 and the light guide bundle 45 that are arranged inside the insertion portion 2 is substantially the same as the arrangement configuration of the first embodiment, a detailed description thereof is omitted here.

(First Reference Example)

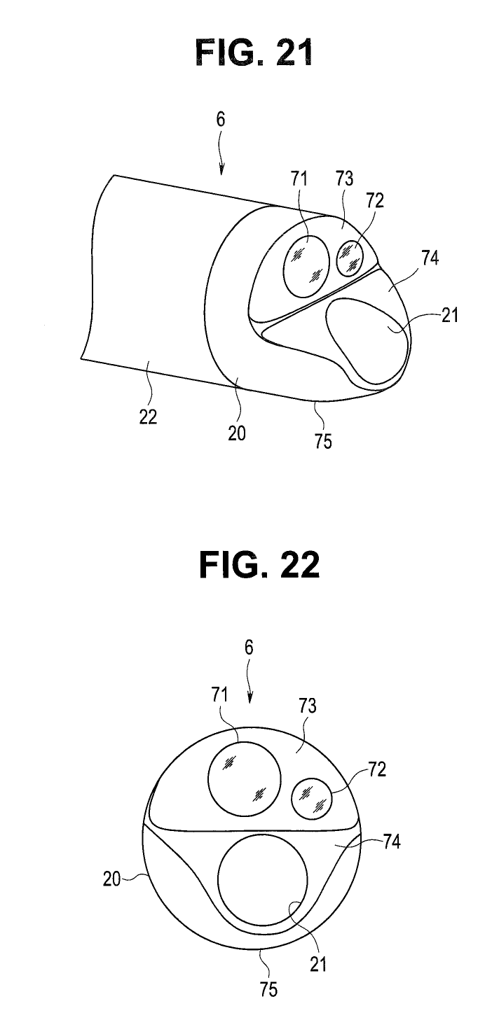

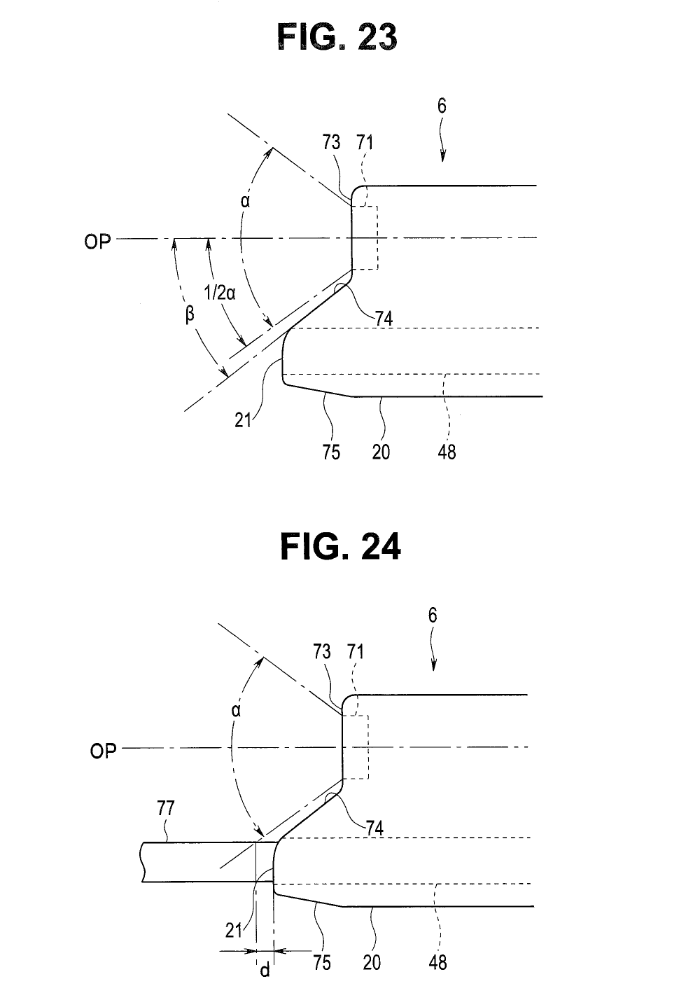

A reference example relating to the configuration of the distal end portion 6 is described hereunder. Note that FIG. 21 is a perspective view illustrating the configuration of a distal end portion, FIG. 22 is a front view illustrating the configuration of the distal end portion, FIG. 23 is a side view illustrating the configuration of the distal end portion, and FIG. 24 is a side view of the distal end portion in a state in which a treatment instrument has been led out from the opening portion.

As shown in FIG. 21 and FIG. 22, the distal end rigid portion 20 of the distal end portion 6 of the endoscope 1 includes a distal end face 73 in which an observation window 71 and an illuminating window 72 are arranged, and a protrusion portion 75 in which a tapered face 74 is limited from the distal end face 73 and which is extended to the distal end side. Note that the opening portion 21 that communicates with the treatment instrument channel 48 is formed in the protrusion portion 75.

As shown in FIG. 23, a predetermined angle of view a for picking up an image of a subject is set in the endoscope 1, and the tapered face 74 formed in the protrusion portion 75 is set at a predetermined angle β with respect to an optical axis OP of photographing light that is incident on the observation window 71.

The predetermined angle β of the tapered face 74 is set to an angle that is equal to or greater than an angle that is a half (½ α) of the predetermined angle of view α (β≧½ α). By adopting this configuration, the protrusion portion 75 does not enter the field of view of the endoscope 1, and furthermore, as shown in FIG. 24, a protrusion length d until a treatment instrument 77 that is inserted into the treatment instrument channel 48 and is led out from the opening portion 21 of the distal end portion 6 enters the field of view is short, and thus the controllability of the treatment instrument 77 can be improved.

(Second Reference Example)

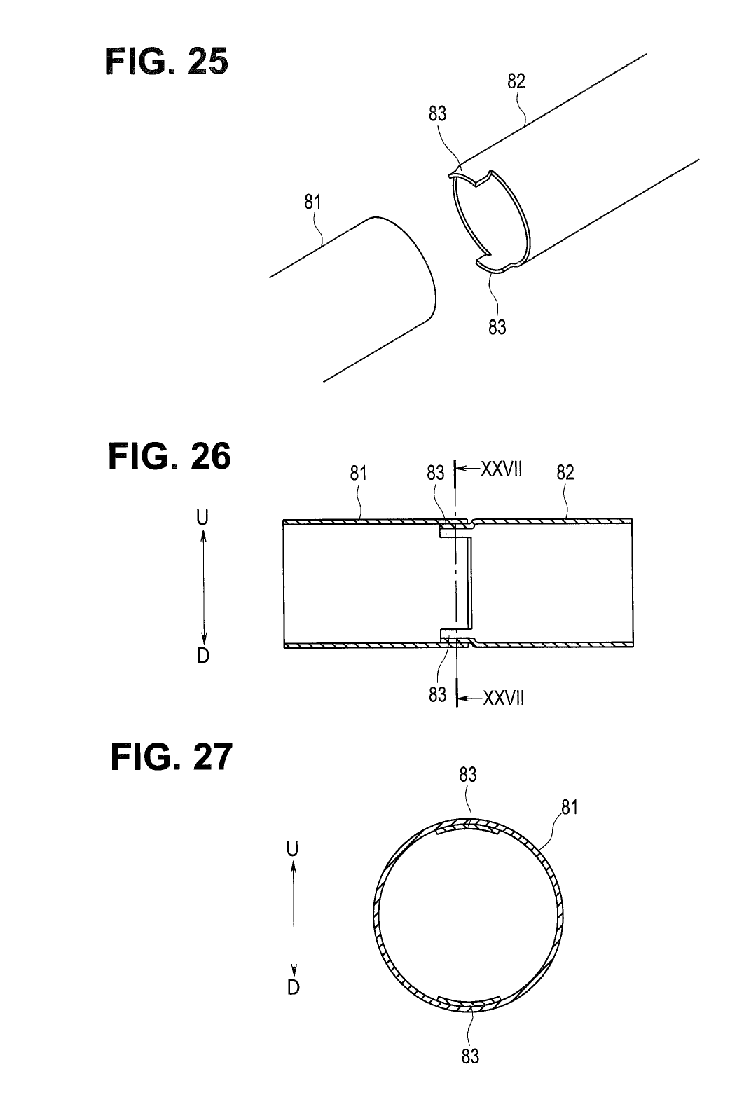

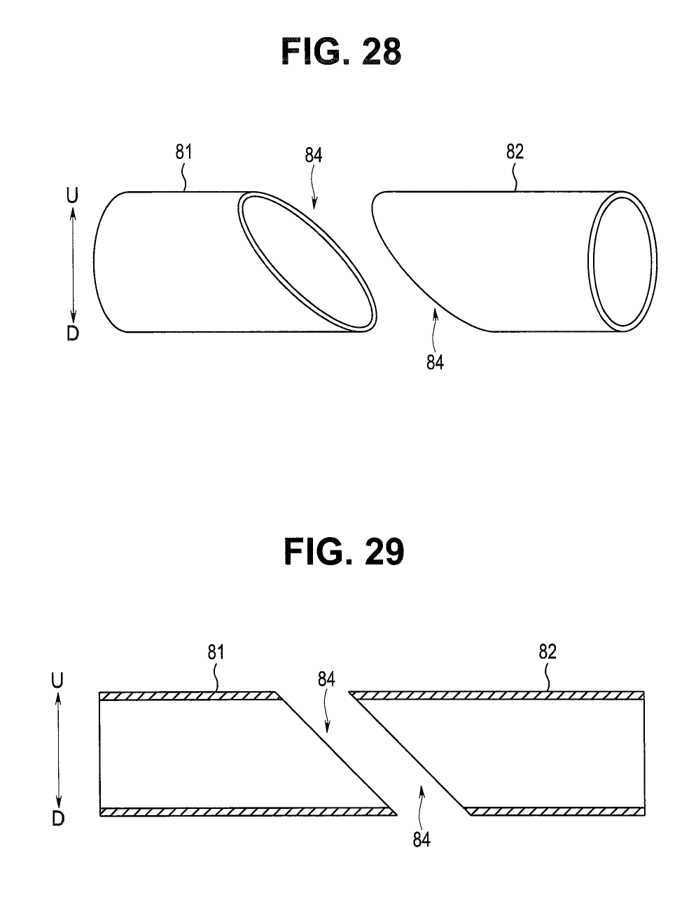

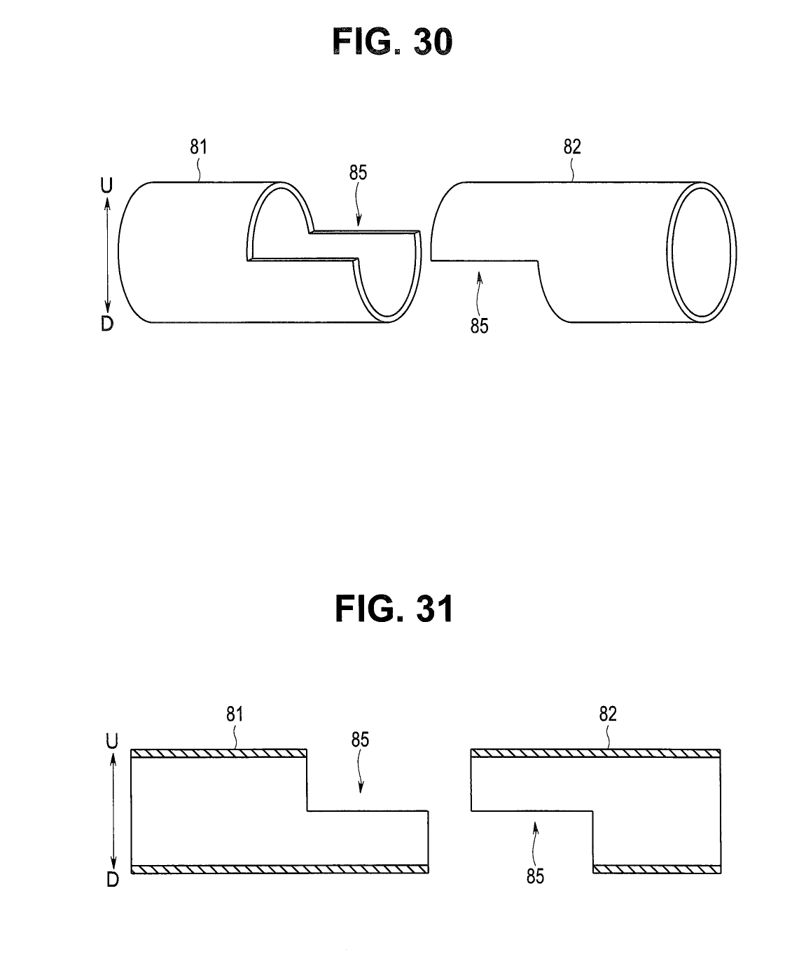

Next, a reference example relating to the connection of cylindrical members arranged in the insertion portion 2 and the like is described hereunder. Note that FIG. 25 is a perspective view illustrating two cylindrical members that are to be fitted together and connected. FIG. 26 is a cross-sectional view of the two cylindrical members that have been fitted together. FIG. 27 is a cross-sectional view of the two cylindrical members along a line XXVII-XXVII in FIG. 26. FIG. 28 is a perspective view illustrating two cylindrical members having a configuration in which connecting end faces are diagonally cut. FIG. 29 is a cross-sectional view illustrating two cylindrical members having a configuration in which connecting end faces are diagonally cut. FIG. 30 is a perspective view illustrating two cylindrical members having a configuration in which connecting end faces are cut in a stepped shape. FIG. 31 is a cross-sectional view illustrating two cylindrical members having a configuration in which connecting end faces are cut in a stepped shape.

As shown in FIG. 25 to FIG. 27, in a configuration in which two cylindrical members 81 and 82 are fitted together and connected, two fitting portions 83 are formed that have a cross-sectional crank shape that is extended so that the second cylindrical member 82 that is the side to fit into fits into the inner circumferential portion of the first cylindrical member 81 that is the side to be fitted into.

In a case where the two cylindrical members 81 and 82 are components constituting the bending portion 7 of the insertion portion 2, the strength in the bending directions can be secured by forming these two fitting portions 83 in conformity with the up-down directions in which the bending portion 7 bends.

Thus, in comparison to a configuration in which the entire circumference of an end portion of the second cylindrical member 82 that is the side to fit into is insertedly fitted into the first cylindrical member 81 that is the side to be fitted into, as in the case of the conventional configuration, by adopting a configuration in which the two fitting portions 83 are formed on the second cylindrical member 82 that is the side to fit into, and insertedly fitting only the two fitting portions 83 into the first cylindrical member 81 that is the side to be fitted into, there is the advantage that space can be secured in the internal space of the fitting portions of these two cylindrical members 81 and 82.

Note that, as shown in FIG. 28 and FIG. 29, a configuration may also be adopted in which connecting end faces 84 of the two cylindrical members 81 and 82 are diagonally cut and inclined, and strength is secured in these inclining directions in accordance with the up-down directions in which the bending portion 7 bends.

In addition, as shown in FIG. 30 and FIG. 31, a configuration may also be adopted in which connecting end faces 85 of the two cylindrical members 81 and 82 are cut in a stepped shape, and strength is secured in the directions of these step portions in accordance with the up-down directions in which the bending portion 7 bends.

The invention described in the foregoing embodiment is not limited to the embodiment and modifications described above, and various modifications can be implemented within a range that does not deviate from the spirit of the present invention in the implementing stage. Further, the above described embodiment includes inventions of various stages, and various inventions can be extracted by appropriately combining a plurality of the disclosed configuration requirements.

For example, if the problem to be solved by the invention can be solved and the described effects of the invention are obtained even after omitting some of the configuration requirements from the entire configuration requirements disclosed according to the embodiment, then the configuration obtained by omitting the relevant configuration requirements can be extracted as an invention.

What is claimed is:

1. An electronic endoscope, comprising:
an insertion portion in which, in order from a distal end thereof, a distal end portion in which an image pickup unit is contained and a metal cylindrical member are connected and made electrically conductive with each other;
a plurality of small signal cables that extend from the image pickup unit and are inserted through and disposed in an inner space formed in the insertion portion;
an operation portion that is connected to the insertion portion and that comprises therein a metal frame that is electrically conductive with the metal cylindrical member;
an endoscope cable having a first end portion that is connected to the operation portion; and
an endoscope connector that is provided at a second end portion of the endoscope cable and that is to be connected to an external device;
wherein the plurality of small signal cables are covered by a metal sheath that is electrically connected to the metal frame and thereby brought together to form a single signal cable bundle and are arranged as the single signal cable bundle inside the endoscope cable as far as the operation portion, the metal sheath is arranged inside the operation portion in a state in which the metal sheath is stripped off inside the operation portion, and the plurality of small signal cables protruding from the metal sheath which is stripped off are arranged in a slackened state inside the operation portion.

2. The electronic endoscope according to claim 1, wherein, with respect to the metal cylindrical member, a plurality of cylindrical-shaped metal members are connected in an electrically conductive manner.

3. The electronic endoscope according to claim 2, wherein, in the inner space, a center of a treatment instrument channel is inserted through and disposed in any one quadrant of an orthogonal coordinate system that takes a center of the insertion portion as an origin, and at least inside the bending portion, centers of the plurality of small signal cables are arranged in other quadrants different from the any one quadrant that are obtained by dividing the orthogonal coordinate system with one axis thereof along upward-downward directions in which the bending portion bends.

4. The electronic endoscope according to claim 3, wherein the plurality of small signal cables are arranged in parallel along the upward-downward directions.

5. The electronic endoscope according to claim 3, wherein:
the centers of the plurality of small signal cables are disposed in a first region obtained by dividing the inner space of the insertion portion with a line that joins two bending operation wires that perform operations to bend the bending portion in the upward-downward directions; and
the center of the treatment instrument channel is disposed in a second region that is different from the first region.

6. The electronic endoscope according to claim 5, comprising:
a light guide bundle that is inserted through and disposed in the inner space and that transmits an illuminating light;
wherein, at least inside the bending portion, a center of the light guide bundle is disposed in the first region.

7. The electronic endoscope according to claim 3, comprising a light guide bundle that is inserted through and disposed in the inner space and that transmits an illuminating light, and that has a center that, at least inside the bending portion, is disposed in a quadrant that is different from the any one quadrant in which the center of the treatment instrument channel is disposed of the orthogonal coordinate system and is also different from the other quadrants in which the centers of the plurality of small signal cables are disposed.

8. The electronic endoscope according to claim 3, wherein:
the plurality of small signal cables are three small signal cables; and
among the three small signal cables, a small signal cable that has a largest diameter is arranged at a center.
